(19) United States
(12) Patent Application Publication
(10) Pub. No.: US 2002/0091371 A1
(43) Pub. Date: Jul. 11, 2002

(54) Salt container system for dialysis apparatus

(76) Inventor: Franz Peter Ritter, Untermeltingen (DE)

Correspondence Address: Klaus J. Bach, 4407 Twin Oaks Drive, Murrysville, PA, 15668, US

(21) Appl. No.: 10/041,967

(22) Filed: Jan. 7, 2002

(30) Foreign Application Priority Data
Jan. 8, 2001 (DE) 101 00 549.0

(51) Int. Cl. A61B 19/00; B65D 75/00

(52) U.S. Cl. 604/408; 206/828

(57) ABSTRACT

In a salt concentrate container system for a dialysis apparatus comprising a salt container consisting of a flexible bag provided at its opposite ends with connecting elements for connection with counter elements of a dialysis apparatus, a rigid bracket extends around the flexible bag and is coupled to the connecting elements at the opposite ends of the bag for firmly holding the connecting elements at a predetermined distance from each other.

### BACKGROUND OF THE INVENTION

The invention relates to a salt container system for dialysis apparatus.

In dialysis apparatus for the dialysis of blood, the salt required in the dialysis apparatus for the preparation of the dialysis liquid is provided in the form of dry concentrated powder in containers in an amount as required for a dialysis procedure. The containers have connections that can be coupled with connections of the dialysis apparatus providing for communication between the dry concentrate containers and a liquid circuit of the dialysis apparatus for using the dry concentrate powder.

Commonly used as such dry concentrate containers are bags which have integrally formed therewith plugs, which can be connected with opposite plug-in structures of the dialysis apparatus. Also, a liquid inlet extends into the interior of the bag and a liquid outlet extends from the bag.

Another common form of such dry concentrate containers are cylindrical rigid cartridges, which have connecting elements arranged at their bottoms and their tops and which are disposed in axial alignment. The connecting elements cooperate with corresponding counter connecting elements of the dialysis apparatus. One of the connecting elements is a liquid inlet and the other is a liquid outlet of the container. The connecting elements of the dry concentrate cartridge are typically provided with membranes, which are pierced, when the connecting elements are joined to the counter connecting elements of the dialysis apparatus in order to establish flow communication.

The dry concentrate containers in the form of flexible bags have the advantage that they can be compressed when they are empty after their content has been used so that they have a small waste volume. They have however the disadvantage that they are relatively expensive to manufacture because the bag itself consists of a flexible plastic material, which is connected to a rigid plug part, which consists also of plastic. In addition, a hose has to be installed in the area of the bag which is remote form the rigid plug. The hose extends into the interior of the bag in order to establish a liquid circulation through the whole bag.

The dry concentrate container in the form of rigid cartridges have the advantage that they can be manufactured inexpensively from a uniform plastic by injection molding. Furthermore, their connecting elements at the bottom and the top are disposed opposite each other so that a liquid circuit is established during use of the containers without problems and without any special measures. However, the rigid containers have the disadvantage that they have a large volume when they are empty after use and therefore represent a relatively large waste volume.

It is the object of the present invention to provide a salt container system for receiving dry concentrate powder for use in dialysis apparatus which has the advantages of flexible bags with respect to a small waste volume after use and also the advantage of the rigid cartridges with connecting elements disposed in axial alignment at opposite ends of the container.

### SUMMARY OF THE INVENTION

In a salt concentrate container system for dialysis apparatus comprising a salt container consisting of a flexible bag provided at its opposite ends with connecting elements for connection with counter elements of a dialysis apparatus, a rigid bracket extends around the flexible bag and is coupled to the connecting elements at the opposite ends of the bag for firmly holding the connecting elements at a predetermined distance.

This container system including a bag and support bracket combination can be inserted into the dialysis apparatus like a cartridge while the possibility that the upper connecting element of the flexible bag is pushed downwardly during closing of the connecting mechanism is eliminated. The support bracket can be re-used with a new dry concentrate bag.

After use only the bag remains therefore as waste which is flexible and takes up only very little space. The two connecting elements are, in contrast to voluminous connectors of the conventional dry concentrate bags, very compact.

The bag itself may have any desired shape; it can be for example rectangular or tetrahedron-shaped. The last mentioned shape has the advantage of permitting very compact stacking of the filled bags.

A particular embodiment of the invention will be described below in greater detail on the basis of the accompanying drawings.

### BRIEF DESCRIPTION OF THE DRAWINGS

FIG. 1 shows a salt container system according to the invention in a support structure of a dialysis apparatus (wherein the dialysis apparatus itself is not shown),

FIG. 2 shows the salt container system in a side view,

FIG. 3 is a front view of the dry concentrate bag,

FIG. 4 shows a first embodiment of the support bracket for the bag,

FIG. 5 is a top view of the support bracket shown in FIG. 4, and

FIG. 6 shows a second embodiment of the support bracket with a dry concentrate bag supported thereby.

### DESCRIPTION OF PREFERRED EMBODIMENTS

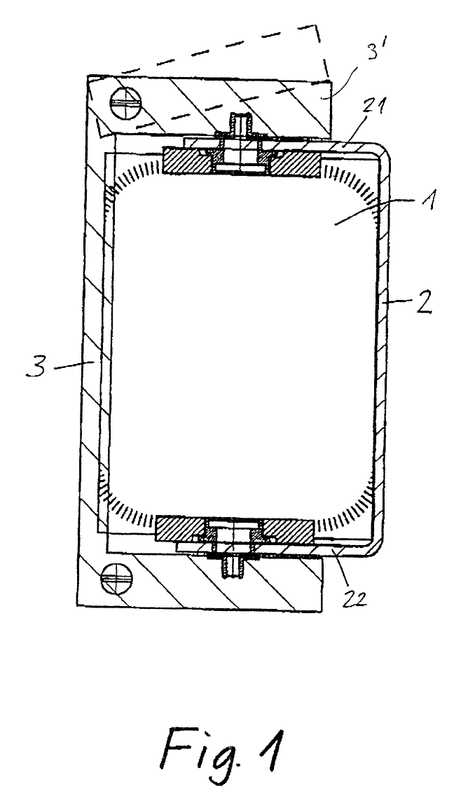

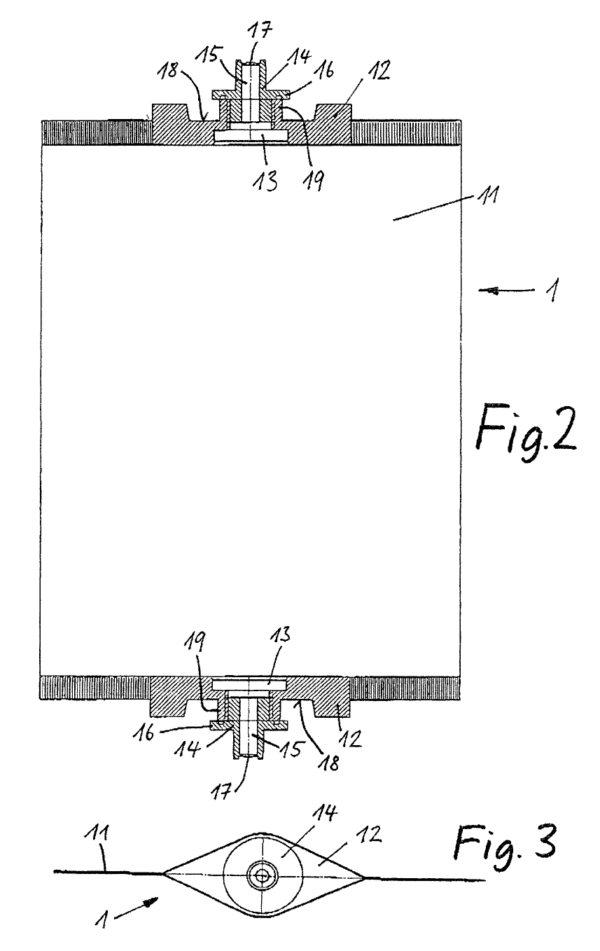

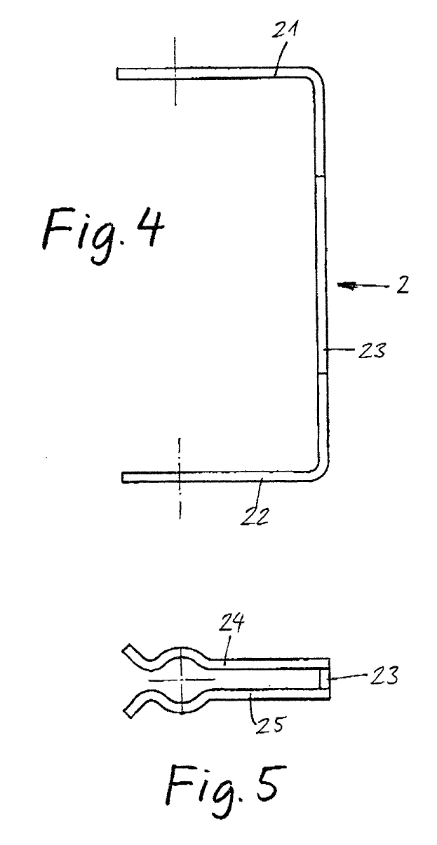

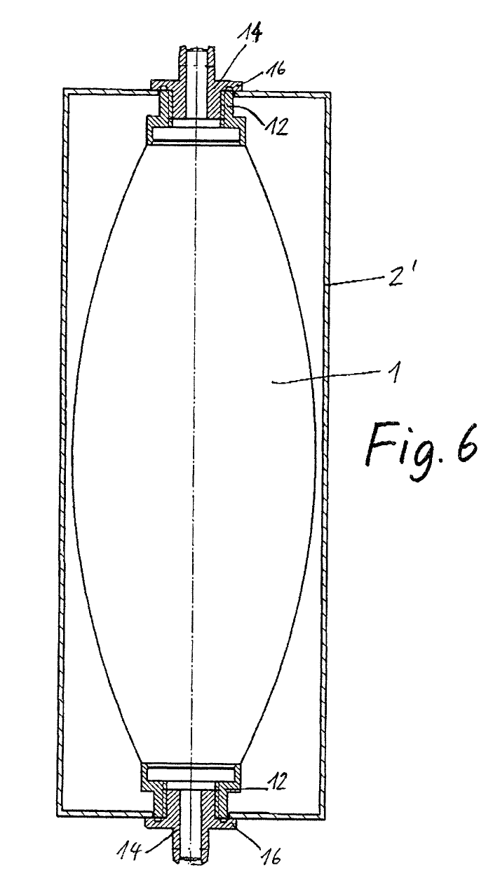

FIG. 1 shows the salt container system according to the invention including a bag 1 as shown in FIGS. 2 and 3 without support structure and a support bracket 2 of the type as shown in FIGS. 4 and 5 disposed in the support structure 3 of a dialysis apparatus which is not shown.

FIG. 2 shows the bag 1 in a front sectional view taken through the connecting elements at the upper and lower ends of the bag. FIG. 3 is a front view that is a top view of the arrangement as shown in FIG. 2. The bag is shown in each case empty wherein the bag is flat.

It is apparent therefrom that the bag body 11 is rectangular in the embodiment. However, the bag may have any desirable shape, it may be hose-like or, as mentioned already earlier, it may be in the form of a tetrahedron.

At both ends of the bag, that is at the top and at the bottom, a connecting element is arranged in the center of the bag. Each connecting element 12 includes a connecting body 12, which is mounted on the bag body 11 and has a center bore 13 with a connecting stub 14 disposed therein. The connecting stub 14 may be threaded or welded onto the connecting element 12 and includes a center passage 15, a disc-like radial flange 5 and a closure membrane 17, which can be pierced. As apparent from the figures, the respective connecting elements 12 are provided in a recess 18 with a cylindrical neck portion 19 which projects outwardly and surrounds the bore 13. The disc-like radial flange 16 of the connecting stub 14 is disposed on the neck portion 19, wherein the radial flange 16 of the connecting stub 14 extends radially beyond the neck portion 19 of the connecting element 12.

FIGS. 4 and 5 show the support bracket 2 for supporting the dry concentrate bag 1 when it is filled and ready for use. FIG. 4 is a side view of the bracket and FIG. 5 is a top view. As apparent from the side view of FIG. 4, the support bracket 2 has a U-shape with upper and lower horizontal legs 21 and 22 and a vertical connecting part 23 extending between the upper and lower legs 21, 22. The center connecting part 23 maintains the horizontal legs 21 and 22 rigidly at a predetermined distance from each other. The two horizontal legs 21, 22 have the same shape—mirror reversed—(so that the support bracket 2 does not require a particular orientation). As apparent from FIG. 5, the two legs 21, 22 are in the form of clip-like clamps, each including two mirror-reversed clamping legs 24 and 25.

For use, the support bracket is assembled with the dry concentrate bag 1 as shown in FIG. 1, wherein the two clamping legs 24, 25 of the upper and the lower support legs 21, 22 extend around the neck portions 19 of each connecting element 12 so as to engage the connecting elements 12 underneath the radial disc-like flange 16 for firm engagement therewith. The rigid support bracket 1 then holds the attached bag 1 in a stretched position so that the upper and lower connecting elements of the bag (both are identical) are supported at a predetermined distance. The support structure 3 of the dialysis apparatus includes a pivotable upper part 3′, which is shown in FIG. 1 by dashed lines in an open position. Upon placement of the support bracket 2 including the bag 1 onto the support structure 3 of the dialysis apparatus and downward tilting of the upper part 3′, the bag 1 with the support bracket 2 is firmly engaged in the support structure 3. At the same time, the closure membranes 17 of the two connecting elements are pierced by piercing members disposed in the support members of the dialysis apparatus. As the connecting elements are firmly engaged and positioned by the bracket 2, they cannot yield while the axial force required for piercing the closure membrane and for providing the sealing connecting between the connecting elements and the support members of the dialysis apparatus is applied.

FIG. 6 shows another embodiment of the support bracket with a dry concentrate bag 1 mounted therein wherein the support bracket 2′ has a tubular a box-like configuration. The support bracket 2′ is closed at the top and the bottom except for a slot for receiving the connecting elements of the bag 1. At one side, the support bracket 2′ is open to permit insertion of the bag 1 whereby the edges of the slots are engaged below the radial flanges 16 of the upper and the lower radial flanges 16 of the connecting stubs 14. In this way, the connecting elements 12 of the bag 1 are supported at a predetermined distance from each other.

What is claimed is:

1. A salt concentrate container system for dialysis apparatus, comprising: a salt container consisting of a flexible bag provided at opposite ends with connecting elements for connection with counter elements of a support structure of a dialysis apparatus, and a rigid bracket extending around said flexible bag and being coupled to the connecting elements at the opposite ends of said bag for holding said connecting elements at a predetermined distance from each other.

2. A salt concentrate container system according to claim 1, wherein said support bracket includes two clamp-like legs rigidly interconnected by a connecting part and including locking means for interconnecting said bracket and said connecting elements of said bag.

3. A salt concentrate container system according to claim 1, wherein said support bracket has the shape of a box, which is open at one side and has end walls at opposite ends, said end walls having slit-like openings for receiving said connecting elements of said bag, with said bag being disposed in said box-shaped bracket.

4. A salt concentrate container system according to claim 2, wherein said connecting elements include neck portions with connecting stubs having radially projecting annular flanges and said bracket legs extend behind said radially projecting annular flanges for engaging said connecting elements.
